(19) United States
(12) Patent Application Publication
(10) Pub. No.: US 2023/0020179 A1
(43) Pub. Date: Jan. 19, 2023

(54) INDWELLING VENOUS CANNULA

(71) Applicant: EBNET MEDICAL GMBH, Schwerin (DE)

(72) Inventor: Jens EBNET, Schwerin (DE)

(21) Appl. No.: 17/786,835

(22) PCT Filed: Dec. 21, 2020
(86) PCT No.: PCT/EP2020/087404

§ 371 Date: Aug. 10, 2022

(30) Foreign Application Priority Data
Dec. 20, 2019 (DE) 10 2019 135 505.2

(51) Int. Cl. A61M 25/00; A61M 25/06; A61M 25/02

(52) U.S. Cl. CPC A61M 25/0017; A61M 25/0045; A61M 25/0606; A61M 25/02; A61M 25/0026; A61M 2025/0266

(57) ABSTRACT

The invention relates to an indwelling venous cannula (1) for application to a living being, having a venous catheter (2), wherein a puncture needle (3) can be guided in a longitudinally movable manner in the venous catheter (2). The puncture needle (3) is designed as a hollow needle over a portion of its length, wherein at least part of an additional portion of the length of the puncture needle (3) is solid.

The invention relates to an indwelling venous cannula for application to a living being having a venous catheter, wherein a puncture needle is longitudinally slidably guidable in the venous catheter.

DE 40 41 720 A1 describes an indwelling venous cannula having a venous catheter and a puncture needle slidable therein, wherein the front, body-penetrating part of the venous catheter is made of stainless steel and the rear part is made of a flexible material, such as a plastic.

It is therefore an object of the invention to create an improved indwelling venous cannula. It is a further object of the invention to create an improved puncture system in the sense of a general puncture system. In principle, it is namely also possible with the improved indwelling venous cannula to puncture all body cavities and body spaces and all anatomical and pathological structures that are to be punctured and to insert a catheter into them in an advantageous manner.

The term “indwelling venous cannula” shall, however, continue to be used in what follows, but it has a broader meaning in the sense of a general puncture system, by means of which it is not just veins that can be punctured. In what follows, the term “veins” thus in principle also encompasses all body cavities and body spaces and all anatomical and pathological structures that are to be punctured and provided with a catheter.

By way of example, the trachea, pleural space, abdominal cavity, intestines, renal pelvis, urinary bladder and bones can be additionally punctured using the indwelling venous cannula according to the invention. In addition, pathological structures such as abscesses in and on the living being can be punctured.

Here, the indwelling venous cannula can either be directly removed after puncturing or else left in place for some time. Arterial blood vessels can advantageously also be punctured.

The object is achieved with an indwelling venous cannula having the features of claim 1. Advantageous embodiments are described in the dependent claims.

In the case of the indwelling venous cannula of the type in question, it is proposed that the puncture needle of the indwelling venous cannula be in the form of a hollow needle over part of the length, wherein another part of the length of the puncture needle is at least partly solid. Such a hollow needle enables the user to aspirate blood even during and after puncturing in order to check directly during and after puncturing whether the relevant vessel has already been correctly punctured. The other part of the length can, however, also be completely solid.

The puncture needle is thus in particular not hollow over the entire length, or part of the puncture needle is solid. Solid can mean in particular that the puncture needle is not hollow. It is conceivable in particular that a near-vein end of the puncture needle is solid, i.e., impermeable to a fluid.

It is possible, but not necessary, for the puncture needle to be hollow only in an upper region. This cavity can, near the vein, be connected to the environment via an upward-pointing hole/opening or via a plurality of such holes/openings.

A near-vein part can in particular be the part that faces the living being. The tip of the puncture needle is, for example, a near-vein part of the puncture needle. Correspondingly, a vein-remote part can be the part that faces the user of the indwelling venous cannula.

Said hole can be a defined distance away from the tip of the puncture needle, which thus does not itself directly lie on a cavity of the puncture needle or does not itself directly merge into such a cavity. The hole can, for example, be arranged on the side of the puncture needle.

This can have the advantage that the puncture needle first has to be advanced a certain distance into the vein so that blood can be aspirated via the puncture needle using an aspiration element.

This increases the certainty of the puncture needle coming to lie in the vein before a venous catheter can be safely advanced over the puncture needle. In the case of commercially available puncture needles, what happens in practice is that the near-vein tip is already in the blood vessel and blood flows back or can be aspirated via the puncture needle, but the near-vein portion of the venous catheter surrounding the puncture needle is still situated outside the vein.

In such a case, if the venous catheter is now advanced over the puncture needle in the direction of the vein without further advancement of the puncture needle, an intended position of the venous catheter is not necessarily achieved. The venous catheter can then, for example, be unwantedly pushed past the vein. This danger can be reduced by the development just mentioned.

Moreover, the risk of carryover of punched-out skin particles into the vein can be reduced, since a puncture needle that is not completely hollow can reduce the risk of a skin cylinder being punched out.

Alternatively or additionally, a plurality of holes can also be present on the puncture needle, which holes can moreover also be arranged in the lateral or bottom region.

The unwanted perforation of the second wall of the vein lying below or behind is nevertheless highly unlikely, since the hole is situated not far away on the portion of the puncture needle that reaches the blood vessel upon advancement thereof.

The hole can, for example, be 0.5-5 millimeters away from the portion of the puncture needle that is nearest the vein. This near-vein portion can be in the form of a tip of a puncture needle having any cut.

In an advantageous development, the indwelling venous cannula can be a catheter system for puncture of the pleural space, the trachea, the urinary bladder or the gastrointestinal tract or for puncture of reservoirs, pump systems, tube systems, pipe systems or port systems.

In an advantageous development, the indwelling venous cannula can be a catheter system for use in the field of interventional radiology, in the field of interventional cardiology or in the field of emergency medicine, disaster medicine or tactical and military medicine.

In an advantageous development, the indwelling venous cannula can be configured for invasive measurement of arterial blood pressure.

The field of application of the indwelling venous cannula according to the invention extends to all common indwelling venous cannulas in human medicine, including pediatric indwelling venous cannulas, and those in veterinary medicine. In particular, the indwelling venous cannula according to the invention is, however, also intended to allow indwelling venous cannulas having larger diameters than conventional indwelling venous cannulas. They can, for example, be classified under a defined inner venous-catheter diameter on the basis of color primers and selected by the operator depending on the application.

At least part of the venous catheter configured for dwelling in the living being can, over its entire length or the predominant part of its length in which the puncture needle is longitudinally slidably guidable, be made of a puncture-resistant material or coated with such a puncture-resistant material.

The puncture resistance of the material refers to possible puncturing of the venous catheter by the puncture needle, which is to be prevented by the puncture-resistant material. The puncture-resistant material can be a metal, including in the form of a metal alloy, or an appropriately puncture-resistant plastic material or natural material. For example, possible plastic materials are carbon fiber-reinforced laminate materials, polymers and/or Teflon, including in combination with one another.

This increases patient safety. Moreover, patient comfort is increased, since skin piercing in particular is painful and it is possible with the indwelling venous cannula according to the invention to actively repeatedly seek out a blood vessel after the skin has been pierced and, while doing so, to remain constantly below the level of the skin in the living being with the near-patient parts of the indwelling venous cannula, in particular with the near-patient pointed end of the puncture needle. This reduces the number of skin piercings required in order to successfully place an indwelling venous cannula. This reduces traumatization of the surrounding tissue. In addition, reducing the number of skin piercings also reduces the number of possible entry points for potential pathogens such as bacteria and viruses through the skin.

To form puncture resistance, use can especially also be made of aramids. This also encompasses, for example, aramid fibers, in particular para- and meta-aramid fibers.

The puncture-resistant material can in particular no longer be present in the (distal) end of the venous catheter that is nearest the patient. The thickness of the puncture-resistant material and/or the material density need not be constant over the entire longitudinal extent over which the puncture-resistant material is present on the venous catheter, but can vary over the length. The puncture-resistant material need not be present everywhere. Gaps can be deliberately left in order, for example, to create a kind of “predetermined bending point”.

The puncture-resistant material can, for example, be formed by a tubular metal body or a metal coating of a base material of the venous catheter, which can, for example, be a plastic material. The metal can, for example, be steel, for example stainless steel, or titanium or, formed therewith, an alloy or a bimetal. In an advantageous embodiment of the invention, the puncture-resistant material is arranged on the inside of the venous catheter, i.e., the puncture-resistant material forms the inner wall of the venous catheter. A protective layer of the venous catheter surrounding the puncture-resistant material on the outside can, for example, be made of plastic.

In an alternative embodiment, the puncture-resistant material is arranged on the outside of the venous catheter. However, it is also conceivable that the puncture-resistant material is arranged both on the inside and on the outside of the venous catheter. The puncture-resistant material can thus form either the inner wall or outer wall of the venous catheter or both. As a result, the improved indwelling venous cannula is more stable and robust than hitherto known indwelling venous cannulas with, at the same time, improved flexibility of the venous catheter. The puncture-resistant material is advantageously made of a material having nonthrombogenic properties. Alternatively or additionally, the puncture-resistant material can be coated with an additional surface having nonthrombogenic properties. This material property is intended to prevent the formation of thrombi (blood clots) on the venous catheter. The puncture-resistant material can be in the form of a metal layer, including as a metal alloy (alloy). Advantageously, the puncture-resistant material is moreover compatible with magnetic resonance imaging (MRI), meaning that the situated venous catheter, or the components of the indwelling venous cannula inserted in the body, remain in the body even during an MRI examination, do not harm it during the MRI examination and moreover cannot dislocate in the magnetic field.

It is advantageous if the venous catheter is not too rigid owing to the puncture-resistant material, i.e., it still has a certain flexibility. Moreover, such materials can be used to make the venous catheter MRI-compatible.

If the indwelling venous cannula should not be completely metal-free- or MRI-compatible, it is conceivable that the indwelling venous cannula is made of materials which are, for example, partially MRI-compatible, for example, i.e., may only be used in certain MRI machines.

In particular, the venous catheter can also be used as a ventilation tube and endotracheal tube. Here, all the advantages described can be used to provide a bite-proof and kink-proof ventilation tube and endotracheal tube. This can, for example, be particularly advantageous for pediatric patients. It is important that a possible helical structure is made of a material which cannot be bitten through and, in the event of nevertheless accidental damage, does not form or yield any sharp or pointed structures. At the same time, it is advantageous if the material of the helical structure is, for example, covered with a softer material or if the helical structure is connected to other materials of the venous catheter. It is also conceivable that the helical structure is only formed over a partial length of the venous catheter or can only be found in a semicircular shape in the wall of same. A venous catheter formed in this way can have, remote from the vein/near to the user, a connector to which a (further) ventilation tube or other airway aids can be connected.

The end of the venous catheter that is nearest the vein can be designed in such a way that it is no longer in the form of an edge-type structure, but rather slightly beadlike. It can also be rimlike, bent inwards or turned inside out. It can also comprise a flexible material or even a foam material or gelatinous material, as well as a less tightly wound helical spring. It can also comprise a particularly atraumatic material.

The puncture resistance has the advantage that the indwelling venous cannula is pierce-resistant, kink-resistant and cut-resistant. This minimizes the risk of damage to the venous catheter. After mispuncture, the indwelling venous cannula can therefore be reused under constant sterile conditions in the same living being, since, even with repeated sliding of the puncture needle against the venous catheter in the longitudinal direction, the latter cannot be damaged by the near-patient tip of the puncture needle. This is a problem with conventional indwelling venous cannulas. Especially as a result of already slight kinking of the flexible venous catheter due to use, the near-patient tip of the puncture needle can cause the venous catheter or parts thereof to shear off, especially when the puncture needle is repeatedly advanced in relation to the venous catheter in the longitudinal direction.

Slidability of the puncture needle in the direction of the vein can be completely or partially prevented by design.

Such a mechanism can, for example, take effect when the puncture needle has already been moved away from the vein from its original position of being as near to the vein as possible. A non-puncture-resistant venous catheter can then no longer be sheared off or damaged in whole or in part by the near-vein tip of the puncture needle, since it can then no longer (and in particular no longer repeatedly) be pushed in the direction of the vein.

All the layers of the indwelling venous cannula that have just been mentioned advantageously have nonthrombogenic properties. Moreover, they advantageously have hypoallergenic and/or antimicrobial properties. All the layers can moreover advantageously consist of a very slippery material or be coated with such a material. Refinement of the layers or the surfaces by means of nanotechnological processes is explicitly possible.

The venous catheter can be made of a self-closing material or material layers. Part of the venous catheter configured for dwelling in the patient can, over its entire length or the predominant part of its length in which the puncture needle is longitudinally slidably guidable, be made of a material or coated with a material that can be punctured, but immediately completely seals itself again after puncturing.

This can be achieved by reinforcing the material with further structures which merely evade a puncturing action and then return to their starting position.

This can also be achieved by the material used having elastic properties. This material can have superplastic deformability as well as high toughness. The material can have a very smooth surface so that fluids can flow with as little turbulence as possible and the surrounding structures, for example the venous wall, are also irritated as little as possible.

In addition, the material can have thrombogenic (clot-forming/clotting-activating) properties, so that a pierced channel is closed by components endogenous to the body, especially blood-endogenous components, for example platelets or fibrin. Here, the venous catheter can consist of multiple layers, with only the inner layers having thrombogenic properties and the outer layers having nonthrombogenic properties. It is also possible for only one layer to have thrombogenic properties and only one further layer to have nonthrombogenic properties. The arrangement of the layers is fundamentally variable, depending on the desired properties of the venous catheter.

What is thus formed is a venous catheter having biological properties, the wall of which self-closes, but which prevents the formation of an excessively large thrombus which could clog the lumen of the venous catheter.

If the venous catheter consists of multiple layers, a kind of “backdrop phenomenon” can also occur, i.e., in the event of unwanted puncturing of the venous catheter, the puncture site is sealed as a result of layers of the wall of the venous catheter sliding against one another and thereby sealing said venous catheter. It is therefore conceivable that the layers are slidable against one another by design.

Advantageously, the material can be a material which does not yield or release any particles or other constituents even in the event of mechanical irritation or other irritation.

The venous catheter and/or the puncture needle can be in the form of a venous catheter and/or puncture needle that unfolds or enlarges owing to body heat. The catheter can undergo an increase in volume owing to body heat. This can cause the outer diameter of the venous catheter to increase. Here, the inner diameter can also increase, but can also decrease.

The venous catheter and/or the puncture needle can also contain, in the wall thereof, an expandable gas or a sterile liquid that causes the abovementioned properties. The venous catheter and/or the puncture needle can also be inflatable or pumpable.

This has the advantage that only a small venous catheter and/or puncture needle, which enlarge in the vein, has to be inserted through the skin without major widening of the skin canal. This can reduce pain and infections.

Venous catheters and/or puncture needles can, however, also undergo a reduction in volume owing to body heat.

The indwelling venous cannula can have at least one holding element for facilitated application to a living being, wherein the holding element has a complementary shape or a negative contour of a human thumb or a human fingertip. As a result, the indwelling venous cannula can be advantageously further developed ergonomically for the user.

The indwelling venous cannula can have a fastening element, wherein the fastening element is configured to fix the indwelling venous cannula to the living being, wherein the fastening element has an adherent or adhesive surface or an adherent or adhesive coating for fixation of the indwelling venous cannula to the living being.

In this way, what can be achieved is that the indwelling venous cannula cannot be accidentally pulled out of the punctured body part of the living being. Moreover, the administration of drugs is also facilitated, since the indwelling venous cannula is held fixed to the patient without being unstable. The indwelling venous cannula can, for example, be fixed to the living being via a self-adhesive wound dressing via the fastening element.

In this way, the indwelling venous cannula can be fixed directly to the skin of the patient after it has been applied. A removable protective film can protect the surface until the indwelling venous cannula has reached the position at which it is to be fixed.

The venous catheter of the indwelling venous cannula can have an undulating surface. An undulating surface is characterized by alternating diameters of the venous catheter at least in the cross section along the longitudinal axis of the venous catheter. An undulation can, for example, consist of a sinus, rectangular, triangular and/or sawtooth wave. Furthermore, it is advantageous if the venous catheter has a helical structure and/or is provided with a helical structure. Owing to the helical structure, the venous catheter has great flexibility which allows facilitated application to the living being. What is thus provided is an indwelling venous cannula which is both cut- and pierce-resistant and flexible.

The venous catheter and/or the puncture needle can have a helical structure and/or be provided with a helical structure, wherein the helical structure has windings, wherein the density of the windings varies over the length of the venous catheter and/or the puncture needle.

Owing to the density of the windings and to the choice of material for the venous catheter, a desired quality, especially with regard to the flexibility and rigidity of the venous catheter, can be achieved depending on the application. For instance, high rigidity with high puncture resistance can be achieved in the case of a tightly wound venous catheter. Flexibility increases if the venous catheter is less tightly wound.

By varying the density of the windings of the venous catheter, it is also possible for segments having less tight winding to be deliberately configured. This gives rise to “crumple zones” in the venous catheter that have greater flexibility. Thus, the venous wall can, for example, be less irritated by the venous catheter. In addition, use can thereby be made of venous catheters which optimally adapt to the course of the vein. Moreover, a venous catheter having “crumple zones” can also optimally adapt to patient movements.

The venous catheter can also have telescoping properties or be in the form of a ramp. It can also change in diameter in the direction of the vein or in the opposite direction.

When choosing the material, recourse can also be made to a combination of different metals or other puncture-resistant materials. It is conceivable that the density of the windings can also be set by the user if necessary, in that the wound-up venous catheter can be pulled apart or pulled together. It is also possible that the density of the windings at different points of the venous catheter is already different by design and thus the flexibility and rigidity of the venous catheter already vary by design, especially in the longitudinal direction.

Similarly, in the region of crossing of the venous catheter into the skin, the venous catheter can be of a different design than in the regions lying further near the vein. For example, it can have an undulating surface or be reinforced with other structures or elements only in this region.

The windings can be formed in such a way that they run parallel or nearly parallel. However, other forms of winding are also possible, including forms with overlapping winding. Windings running in opposite directions or windings having different angles of inclination are conceivable, too. Especially as opposed to parallel windings, windings having an angle of inclination are wound at an angle, i.e., the windings are not oriented substantially orthogonally to a longitudinal axis of the windings, but at an angle of inclination with respect to the longitudinal axis. Moreover, meshlike structures are possible.

The venous catheter can also be designed with one or more removable layers. If they are removed, the inner diameter and/or the lumen of the venous catheter increases. In this way, layers of the venous catheter colonized by pathogens or provided with blood clots can be easily removed. However, it is also possible that, in this way, layers can be introduced from the outside and the venous catheter can thus be supplemented with further layers.

The venous catheter can, for example, contain multiple inner tubes which can be removed if necessary.

However, the inner tubes can also be designed with a very thin wall, so that they can be removed without any major change in the inner diameter of the venous catheter. Here, they can be filmlike. It is also possible to speak of “onion-skin-like” in this connection. As a result, the inner wall of the venous catheter can be repeatedly provided with a new wall which has contact with fluid and blood.

The venous catheter can also consist of a resorbable or dissolvable material, for example a carbohydrate, or be coated with such a material over its entire length or a partial length. This material can give the venous catheter a certain degree of hardness, and such a material can also make the near-vein tip hard, so that it is easily advanceable into the vein. Upon contact with blood, the material is resorbed, i.e., dissolved in the blood. As a result, less hard structures can be uncovered. Thus, a venous catheter can be hard before insertion into the patient and especially before insertion into the vein and can then become soft.

An outer resorbable or dissolvable (referred to hereinafter as just “dissolvable”) layer or substance around the venous catheter, which layer or substance can also have an antimicrobial effect, can also be advantageous because a venous catheter is contaminated by pathogens when inserted through the skin despite sterile conditions, which pathogens are, for example, located directly under the skin and are not accessible for skin disinfection. If a pathogen-contaminated outer layer of the venous catheter now dissolves in the patient and especially in the blood, what can occur is exposure of the patient to pathogens for a limited period of time as a result of pathogens being washed into the bloodstream, for example. However, they are not able to remain on the venous catheter for a relatively long period of time, where they can multiply and form biofilms, for example. Because of the blood flow, the positive effect just mentioned can be particularly pronounced in the subregion of the venous catheter that is surrounded by blood flow. The venous catheter can be surrounded by an outer dissolvable layer over a partial length or its entire length.

Similarly, the puncture needle can be made of a resorbable material and become soft after insertion into the patient and the vein. In such an embodiment, the puncture needle itself can constitute the venous catheter. It is then possible for the puncture needle to be designed like a venous catheter and to have corresponding properties. The near-vein end of this modified puncture needle can then be made of a hard dissolvable material which forms the tip. It is also possible to form a cut, for example a triangular cut. Upon contact with the patient and especially the vein, the material is dissolved and uncovers the other portions of the puncture needle. The puncture needle can now be used directly as a venous catheter. Here, the dissolvable material can also initially block the interior of the puncture needle and thus ensure that no particles or, for example, no punched-out skin cylinders are carried over into the vein.

It is conceivable that all the components of the in indwelling venous cannula consist of a dissolvable material or are coated with such a material. The dissolvable material can, for example, be a carbohydrate, especially glucose, sucrose, amylose and/or starch.

The near-vein end of the puncture needle, especially the end which is sharp or provided with a cut, can consist of a resorbable or dissolvable material, for example one of the above-described carbohydrates, or be coated with such a material it dissolves in the patient or in the bloodstream. In this way, the puncture needle becomes blunt when it is situated in the patient. It is also possible that the fluid flow through the puncture needle or, analogously, the fluid flow through a venous catheter is interrupted or blocked as a result of rapid dissolution of the resorbable material. As a result, unwanted administration of a fluid into a blood vessel can be avoided. This may be relevant if a fluid or a substance, for example, must only be administered under the skin (subcutaneously).

A conventionally designed indwelling venous cannula may no longer be necessary, since the puncture needle, owing to its reabsorbable properties, already forms the venous catheter and can be left in the vein for use.

The venous catheter and/or the puncture needle can, for example, be wound up like a helical spring made of a metal; the individual layers can lie closely next to one another, so that they are in contact with one another. This is similar to the structure of the Seldinger wire, which is already known and which consists of a tightly wound steel wire. Owing to the tightly wound layers, the venous catheter is cut- and pierce-resistant and, at the same time, flexible.

Owing to a helical spring and/or other structures reinforcing the venous catheter, said venous catheter is especially also suitable for applications which involve increased flow rates and increased infusion pressure, for example when infusing contrast media in the field of radiology.

The helical spring can also be designed in such a way that a kind of “Windkessel effect” arises, i.e., the helical spring and venous catheter can compensate for pressure fluctuations through elasticity. This can also prevent or at least minimize the unwanted delivery of relatively large amounts of liquid within a short time. It is conceivable, for example, that the stated property of the helical spring, for example expansion or enlargement of the diameter, uncovers at least one opening via which the unwantedly inflowing fluid can now be discharged. Also possible is a kind of temporary storage of the excess fluid that has flowed in in the venous catheter itself with later removal by the user.

It is also possible for the helical spring to be designed in such a way that, after the venous catheter has been inserted into the vein or after said venous catheter has reached its desired end position, said helical spring is removed, i.e., it can, for example, be pulled out of the venous catheter in the longitudinal direction away from the vein.

Here, it is advantageous if the helical spring detaches or is detachable from the venous catheter owing to specific material properties of the helical spring itself or to those of the rest of the venous catheter. These can be thermoplastic properties, for example, but the helical spring can also be slippery.

This gives rise to a venous catheter which is easily insertable into the vein in a sufficiently rigid state thanks to the helical spring, but, after the desired end position has been reached and after the helical spring has been removed, does not constantly irritate the venous wall through excessively high rigidity, which can lead to complications.

The puncture needle can be designed according to a Seldinger wire, but additionally with a puncture tip Said wire is inserted into a vein or into some other anatomical structure of a patient and, via said wire, a catheter can be introduced into the patient.

A Seldinger wire can accidentally remain in a patient as a complication if removal thereof is forgotten following the insertion of a catheter which uses said Seldinger wire internally as a guide wire and thus as a guide rail.

Such a complication has been described repeatedly in the medical literature.

It is not necessarily prevented by a classic Seldinger wire owing to the design, since said Seldinger wire has no structure at its vein-remote end that prevents the Seldinger wire from being unintentionally inserted too deeply into a catheter and/or into a vein and then possibly being forgotten therein.

A new Seldinger wire is proposed, which, at its vein-remote end, can be advantageously further developed with the following features:
a) The vein-remote end can be curved, angled, hooked, looped, J-shaped or U-shaped or inversely J-shaped or U-shaped. Here, it is advantageous if the vein-remote end is very flexible and user-friendly, so that a catheter can be quickly slid over it. If the catheter has then been slid on, the vein-remote end temporarily assumes the shape of the catheter, which shape is generally straighter. If the full length of the catheter now passes the even longer Seldinger wire, it reassumes at its vein-remote end the shape described in the first sentence of a). Now, the vein-remote end of the Seldinger wire can no longer pass through the vein-remote end of the catheter, since it does not fit in here owing to the shape. Thus, in the case of a looped design or other design, what can occur for example is that the shape of the inner side of the loop, which is then concave with respect to the situated catheter, ensures that the thus modified Seldinger wire can only be pushed through the venous catheter using an active bending movement. An unwanted disappearance of the Seldinger wire in the venous catheter is prevented by the inner side of the loop of the Seldinger wire meeting the vein-remote end of the venous catheter.
   Under a), the description is not at all of just a reversed conventional Seldinger wire which already has a J-shaped structure at its near-vein end.
b) The vein-remote end of the new Seldinger wire can have attached thereto an element which protrudes from the wire and which must be pressed down actively by the user so that a catheter can be slid onto the Seldinger wire. Said element can be provided with a spring mechanism and can also be rectangular, in the form of a container, in the form of a stamp, or in the form of a ramp. If the catheter is now situated over said element and surrounds the Seldinger wire at this point, the element is pushed in the direction of the wire and the catheter can be pushed over the wire. If the vein-remote end of the catheter now passes the element, it returns to its starting position and the Seldinger wire can no longer completely disappear in the catheter and thus in the vein purely passively.
c) At its vein-remote end over a defined length, the Seldinger wire can also be wound differently or have a larger diameter. It can also be particularly compressible or expandable over a defined length.
d) What can be attached via a connector, or else without a connector, is a kind of stopper or some element which increases the circumference of the vein-remote end of the Seldinger wire, which is then attached when the catheter has passed the wire in the direction of the vein. What can also be concerned here is a cylindrical or disklike element having an inner cavity through which the Seldinger wire is guided.

However, it is also conceivable for the venous catheter to have grooves distributed over the circumference, such as a corrugated tube, which grooves provide an undulating surface. A high degree of flexibility of the venous catheter can thus be achieved. The grooves run continuously over the entire circumference of the venous catheter and are advantageously parallelly arranged at regular or irregular distances over the length of the venous catheter. The venous catheter thus has a varying diameter. In this way, it is possible for the venous catheter to be orientable in different positions. However, it is also conceivable that the grooves can extend with a helical distribution over the circumference. Such helical grooves have the advantage that the pressure loss of a fluid conducted through the venous catheter is reduced and, at the same time, turbulence of the fluid can be achieved.

The venous catheter can be advantageously further developed with one or more reinforcement layers. What can be achieved thereby is that its lumen/inner diameter is no longer compressible/squeezable in any way by external influences. The venous catheter can now also be directly securely sewn on using a thread without possible “constriction” of the venous catheter.

Owing to the abovementioned properties of the indwelling venous cannula, it may, however, also be possible for a puncture needle withdrawn in the venous catheter to be initially left in the venous catheter in order to reperform a puncture at a later time, for example a puncture of deeper structures.

The venous catheter can have a sensor at a near-vein end. Via the sensor, it is possible, for example, to collect data of the living being, such as blood pressure. It can be electrically conductively or data-transmittingly connected to components of the venous catheter, for example a helical structure. In an advantageous embodiment, the helical structure can already be in the form of a sensor.

It is also possible for the sensor to have a flat or undulating structure.

The helical spring can, however, also be electrically nonconductive or consist of an electrically nonconductive material. It is also possible to coat the helical spring with an electrically insulating material.

What can also be attached to the near-vein end of the indwelling venous cannula, especially the venous catheter, is a sensor which detects when the distance between the venous wall and the venous catheter is too small. In this way, the position of the venous catheter or the entire indwelling venous cannula can be corrected, possibly even automatically corrected. It is conceivable that said sensor also has an alarm function, by means of which a warning can be given of blood clots or other structures, for example bacterial clusters.

This can also prevent or at least reduce the risk of further traumatization and also, for example, unwanted puncturing of the venous wall in the case of a venous catheter already in the desired position.

A clamping element can be arranged on the venous catheter, wherein the clamping element is configured to interrupt a fluid flow through the venous catheter. In principle, such a clamping element can be already permanently integrated on all components of the indwelling venous cannula.

The venous catheter of the indwelling venous cannula can have, at a near-patient end, a dilation body for uniform expansion of a punctured body part. This has the advantage that the venous catheter can be inserted into the punctured body part of the living being without additional widening of the puncture site, by the dilation body over the venous catheter, or a dilation body integrated into the venous catheter, being pushed together with the venous catheter over the puncture needle into the punctured body part, whereupon the necessary dilation is effected and the venous catheter can be pushed further into the body part.

The dilation body can taper at an acute angle of less than 11 degrees, based on a 360° system. The angle refers to the angle measurement between two outer dilation faces of the dilation body and not to the central axis of the dilation body.

Advantageously, the dilation body can taper at an acute angle of less than 10.5 degrees. This has the advantage that the venous catheter can be inserted into the punctured body part easily and without additional widening of the puncture site, by the dilation body being pushed over the puncture needle into the punctured body part, whereupon the necessary dilation is effected and the venous catheter can be pushed into the body part. This requires that the dilation body taper at an acute angle of less than 11 degrees, in particular less than 10.5 degrees, so that advancement through the skin can be effected during the dilation process. It is conceivable that the dilation body consists of a material or is coated with a material that reduces frictional resistance in order to facilitate advancement of the dilation body. It is also conceivable that, after the dilation body has been inserted into the punctured body part, it is widened and thus the puncture site widens in such a way that the venous catheter can be pushed into the punctured body part.

It is also possible for the dilation body to be in a form which allows stepwise widening of the vein and/or a puncture site and/or a puncture channel (referred to hereinafter as just “puncture channel”).

To this end, the dilation body can be designed in such a way that its diameter changes, for example increases, from near the vein to remote from the vein. This can occur continuously or stepwise. In the latter case, the diameter is initially constant over a certain distance before a jump in caliber occurs. This sequence can be repeated multiple times until the vein-remote end of the dilation body has been reached.

To increase patient safety, it can be advantageous here if, for each jump in caliber, the location of the jump in caliber contains an element indicating the location of the jump in caliber. Said element can also be designed in such a way that it encompasses, for example also circularly surrounds, the dilation body at the location of the jump in caliber. If a jump in caliber is now to take place as part of the dilation process, said element must first be overcome or be pushed by the user to another location of the dilation body. This ensures that the jump in caliber is noticed by the user. Said user can now decide whether further widening of the puncture channel is necessary.

An aspiration element or else a tube element can also be connected to the vein-remote end of the dilation body, optionally via a connection element or extension element. If the dilation body is hollow, the correct position in the vein can be checked when widening the puncture channel by using an aspiration element, for example a syringe, to aspirate or suck blood via the dilation body. This can be done continuously if a guide wire is not used, which completely fills the lumen of the dilation body. However, it is also possible to use a guide wire which is hollow on the inside and via which blood is aspirated. It is also possible to first insert the dilation body with the aid of a guide wire, to then remove said guide wire later on, and to then advance the dilation body even deeper into the vein under constant aspiration.

For continuous verification of the correct position of the venous catheter in the blood vessel, blood can be aspirated via the situated dilation body, either constantly or as required, when widening the puncture channel.

A dilation body designed in this way allows gentle stepwise widening of a puncture channel by means of a single dilation body without having to sequentially slide multiple dilation bodies having increasing diameters onto a guide wire and to remove them again.

If a puncture needle is situated in a venous catheter, it is also possible for a puncture needle projecting beyond the venous catheter in the longitudinal direction toward the vein to be in the form of a dilation body and to have the abovementioned properties.

A further structure can, for example, also be in the form of an applicator or sponge or foam ring or gel ring that partially or completely surrounds the components of the dilation body and wets them with a friction-reducing or antimicrobial substance before or during insertion into the vein.

The venous catheter of the indwelling venous cannula can have, at the near-patient end, holes distributed over the circumference for homogeneous supply of a fluid to the living being. This has the advantage that, for example, the administration of a drug into the body of the living being can be carried out more homogeneously than in the case of conventional indwelling venous cannulas, meaning that the drug is not undesirably concentrated in one place. Moreover, the removal of, for example, blood or other liquids is more easily possible, as is drainage of, for example, secretions or air, depending on the particular application.

The venous catheter can be curved or angled, especially at the end nearest the vein. The venous catheter can also be J-shaped, in which case it can be flexible especially in the region of the J-shape, and also more flexible than at its other sections. It can, however, also be U-shaped. This property may only become noticeable when the venous catheter is advanced over the puncture needle, since the venous catheter fits snugly in the starting position of the straight puncture needle and thus, in the starting position, the puncture needle splints the venous catheter designed in this way.

The thus curved or angled near-vein end of the venous catheter can be designed here in such a way that, when the venous catheter is advanced over the puncture needle, a desired direction of advancement is specified. Moreover, in the embodiment just mentioned, the near-vein end of the venous catheter never comes into direct contact with the wall of the blood vessel if a desired position in the blood vessel has already been reached. The venous wall can therefore not be unwantedly punctured so easily. The curve or bend can be neutralizable here by external influences.

The holes of the venous catheter can be arranged here at the point at which the bend or curve of the venous catheter is at its maximum. As a result, liquids to be infused, for example, continue to follow the direction of flow away from the venous catheter into the blood vessel.

The holes can also be in the form of flaplike elements (referred to hereinafter as “flaps”) and/or be connected to such elements. Here, it is advantageous if the flaps exert a valve effect and/or control the flow of fluids in an advantageous manner. Thus, the flow of fluids can also be deflected by the flaps.

Unlike conventional indwelling venous cannulas, the venous catheter of the indwelling venous cannula according to the invention can have not only a single-lumen design, but also at least two lumens separate from one another. The puncture needle is guided through one of the lumens. This gives rise to a multilumen indwelling venous cannula through which different drugs and solutions can be simultaneously administered separately from one another. Mixing of different drugs and solutions can be avoided by means of holes or exit holes arranged on the side of the venous catheter. Moreover, the flow rates of the administered liquids can be increased by provision of multiple lateral holes or exit holes. Pressure infusions can be advantageously used. The structure of the above-described puncture-resistant material can also be adapted here to the existence of the lateral exit holes, for example annularly around the exit holes, in order to additionally stabilize them or keep them open.

It is also conceivable that the holes on the venous catheter are closable by flexible structures which can be operated in a user-friendly manner by the user. Springlike, cablelike or stentlike mechanisms can be used here.

It is also possible for classic holes directly visible to the human eye to be no longer present on the venous catheter or on the indwelling venous cannula. The venous catheter can nevertheless be partially or fully further developed by, or coated with, a material permeable to fluids, gases or vapors. It is possible that said material has meshlike or gridlike structures and therefore exhibits permeability. Similarly, it can have very small holes, for example also pores. These structures can all be also very small. For example, nanostructures can be used. It is also possible to use structures which are only selectively permeable only to certain fluids, gases, vapors or substances.

In the case of the multilumen design of the indwelling venous cannula, it is possible that the holes/exit holes can also be arranged at the top and bottom of the venous catheter. In the case of a multilumen design of the indwelling venous cannula, multiple connection elements for connection of an aspiration element or multiple aspiration elements are also advantageous. This allows improved checking of the position of the indwelling venous cannula or the venous catheter in, for example, a blood vessel, since the various sections of the venous catheter can be checked for their position in, for example, a blood vessel. This is, for example, of medical relevance when puncturing deeper blood vessels. If the lumen which has its opening in the blood vessel near the user is checked for the retrograde movement of the blood, it can generally be assumed that all other lumens also end in the blood vessel, since the openings thereof are arranged even deeper in the blood vessel. The various openings can also each be lined with or surrounded by a material visible in an X-ray image for exact checking of the position of the openings and thus also for the exact checking of the position of the indwelling venous cannula or the venous catheter. The connection element or the connection elements can be arranged on the indwelling venous cannula in such a way that they point away from the skin of the patient at a certain angle, for example at a 90 degree angle or at a 45 degree angle.

In one embodiment of the indwelling venous cannula in the multilumen design, it is possible, for example, for only the part of the venous catheter in which the puncture needle is longitudinally slidably guidable to comprise the puncture-resistant material.

In the case of the multilumen design of the indwelling venous cannula, the lumen through which the puncture needle is guided in the venous catheter can preferably lie centrally between the other lumens.

This simplifies puncturing, since the direction of puncturing can be easily assessed by the user, since puncturing can be carried out in a straight line.

In the case of an at least two-lumen design of the indwelling venous cannula, the venous catheter can have an outer lumen which partially or completely surrounds all the other lumens on the outside in a circular or semicircular manner and/or lies under the outer wall of the venous catheter. Said lumen can, then, have holes arranged over a partial length or the entire length of the venous catheter, via which holes a fluid can be administered once, repeatedly or continuously.

Said lumen is primarily intended for supply of fluids or substances, for example antimicrobial and/or thrombus-dissolving substances, which are to wet the venous catheter from the outside or to act very generally thereon from the outside and on its environment.

For example, deposits on the venous catheter can be removed or rinsed away. In addition, pathogen colonization can also be prevented or at least reduced. Said lumen can be specially marked on the indwelling venous cannula, since its holes are not necessarily able to come to lie in the vein. A rinsing solution can also be continuously administered via the lumen; it can then be referred to, for example, as the “rinsing lumen”. It is also possible for the holes of said lumen to be directly adjacent to the other holes of the venous catheter, so that fluids and substances can be administered in the immediate vicinity thereof.

By means of the indwelling venous cannula, it is possible to introduce a local anesthetic and/or antimicrobial substances into the body of the patient, either continuously or for a limited time. In this way, a reduction in pain when inserting the indwelling venous cannula or leaving it in place is realizable for example.

Over the entire length or at defined points, the indwelling venous cannula or the venous catheter can contain or be coated with a material which is visible in an X-ray image and thus allows exact checking of the position of the indwelling venous cannula or the venous catheter in the body.

The venous catheter of the indwelling venous cannula can have a connection element for connection of an aspiration element, such as a syringe, wherein the venous catheter has a structure, wherein the structure is configured for locking with the aspiration element. As a result, a check can be made at any time by aspiration to determine whether the venous catheter is situated in the target structure of the living being. For example, by aspiration of blood, it is possible to verify whether the venous catheter of the indwelling venous cannula is still situated in a blood vessel. Furthermore, such a connection element allows simplified drug administration.

A connection element at the vein-remote end of the indwelling venous cannula for connection of an aspiration element, for example a syringe, can be designed in such a way that it contains not only a conventional element for undoing or inserting an aspiration element, but also an element having an additional securing mechanism.

Such a securing element ensures reliable prevention of disconnections between the indwelling venous cannula and the aspiration element and/or other elements connected to the indwelling venous cannula (referred to hereinafter as “aspiration element”), for example infusion tubes.

The securing element can have a stop which prevents rotation or unscrewing of an aspiration element from the indwelling venous cannula.

The stop can interact with at least one travel-limiting element.

The interaction of the stop with the travel-limiting element provides a fixing device, wherein a maximum angle of rotation of the fixing device of the aspiration element limits.

If a stop attached to the aspiration element comes into contact with the travel-limiting element, the aspiration element can no longer be further rotated in the particular direction of rotation.

Multiple travel-limiting elements can also be present, for example at least two travel-limiting elements. They can be shaped and operable in different ways (e.g., depressible). In particular, they can also have a locking function. Here, the travel-limiting element can, however, also be designed in such a way that it is connected to the indwelling venous cannula via a helical spring. Here, the helical spring can be countersunk in a cylindrical recess/a hole in the indwelling venous cannula. This makes it possible to actively push the travel-limiting element in the direction of the indwelling venous cannula and to thus temporarily actively cancel the effect of the travel-limiting element in order, however, to be able to actively rotate a stop over the travel-limiting element. The travel-limiting element itself can, by way of example, be in the form of a stamp or a ramp, but other forms are also conceivable.

However, it is also conceivable that an aspiration element snaps into a structure of the indwelling venous cannula, thereby making disconnection impossible. Moreover, it is advantageous if said structure can differentiate between a venous position or function and an arterial position or function.

A mechanism can also be designed that automatically interrupts a fluid flow if there is no aspiration element on the indwelling venous cannula.

It is conceivable that the venous catheter is advanced over the inner puncture needle in the direction of the vein as a result of rotations of, for example, a connection element or another component of the indwelling venous cannula. To this end, use can be made of intermeshing gears or gearlike elements (referred to hereinafter as “gear”/“gears”).

This can be advantageous for difficult venipunctures when the indwelling venous cannula and especially the puncture needle have to be stabilized using one hand and the venous catheter has to be advanced precisely over the puncture needle in the direction of the vein using the other hand.

Specifically, the connection element can be arranged on the upper side of the indwelling venous cannula in a region which cannot be inserted into a patient.

Nevertheless, an aspiration aid, for example a syringe, can be connectable via the connection element. The connection element can be hollow on the inside and be designed with a cover which is connected to the connection element via a tab.

In order, then, to form the abovementioned mechanism, the rotatable connection element, for example at its lower section, can be let into the cavity of the indwelling venous cannula, which forms the extension of the venous catheter in the longitudinal direction away from the vein in the direction of the user.

The lower section of the rotatable connection element can be further developed with a gear. Similarly, the venous catheter can run up to said section or beyond said section in the longitudinal direction away from the vein in the direction of the user and be provided with a gear.

This can, then, engage in the gear of the connection element; with regard to the arrangement/orientation of the gears in relation to one another, different angles are conceivable. When the connection element is rotated about its own longitudinal axis, the venous catheter is, then, slid in the longitudinal direction. In an alternative or additional embodiment, the venous catheter can also be rotated about its own longitudinal axis. However, it is also conceivable that, by analogy, the puncture needle can be moved correspondingly.

The puncture needle can also be in the form of a curved and/or angled hollow needle. In particular, it can also be helical spring-shaped. The venous catheter surrounding the puncture needle can have sufficient flexibility and robustness in order to also surround an unstraight puncture needle and adapt itself in terms of its shape. Thus, the venous catheter can also be curved, angled and/or helical spring-shaped in the starting position before, during or after use on the patient.

In the case of the helical spring-shaped design, the components designed in this way and to be introduced into the patient are introduced into the patient by means of rotation. Here, the axis of rotation runs orthogonally to the skin surface.

Similarly, the entire indwelling venous cannula can be curved, angled and/or helical spring-shaped. However, it is also possible that especially the vein-remote components of the indwelling venous cannula continue to be straight.

It is also possible for the puncture needle and/or venous catheter to contain straight sections between curved, angled or spring-shaped sections. It is also possible for curved, angled or spring-shaped sections to be combined with one another as desired. A screwlike structure of all the components just mentioned is also conceivable.

If, then. a venous catheter is advanced over the puncture needle in the direction of the vein, it can also reassume its original shape, for example a linear form. It can also assume a specific shape, or steer in a specific direction by design.

It is conceivable for a curved, angled and/or helical spring-shaped puncture needle, on its own or in combination with a venous catheter in an indwelling venous cannula, to be used where it is not absolutely necessary to be positioned in a vein. For example, use as a subcutaneous catheter, injection and/or infusion system in palliative medicine is conceivable. In this field, the subcutaneous administration of drugs and infusion solutions is very important.

At its near-user end, a subcutaneous catheter system designed in this way can merely be designed with a connection element for an aspiration element, for example a syringe. It is also conceivable that only one infusion line can be attached to the vein-remote end, for example via a plug-in or rotary connection and/or via a connector of some other design.

Although possible, the puncture needle need not consist of a metal. For example, it can also consist of a plastic material having sufficient hardness. This can advantageously have thermoplastic properties. Thus, it can become softer when it is advanced into the vein or is situated in the vein.

The puncture needle can also sectionally consist of different materials and/or not be hard over a defined distance.

In such a design, the puncture needle can have, at the near-vein end and distributed over the circumference, one or more arranged holes through which a homogeneous delivery of a fluid, for example a drug or an infusion solution, into the patient can be achieved. Similarly, blood can be collected through the holes.

An expansion body for fixing the position of the venous catheter in the punctured body part can be arranged on the venous catheter of the indwelling venous cannula.

Such an expansion body can, for example, be in the form of an inflatable cuff, which is arranged, for example, on the outside of the venous catheter. Such a cuff can be used to seal off the indwelling venous cannula from the outside by inflating the cuff introduced under the skin or filling it with liquid and thus sealing the puncture system from the outside and also advantageously fixing it in its position.

However, such an expansion body can also be formed on the inside of the venous catheter and, if necessary, line the inside of the venous catheter and, for example, seal and/or block it against blood flowing back.

The expansion body can be in the form of a cuff or else a sleeve, for example a Dacron sleeve. Through via a small tube or catheter of its own which can, for example, be integrated into or guided along the venous catheter, it can also be fillable with a fluid and/or a gas and/or vapor via a connection element having an aspiration element, for example a syringe.

A pressure gauge can also be connected to the connection element. Here, the connection element is advantageously further developed with a valve, and this prevents the fluid and/or the gas and/or vapor from escaping spontaneously and retrogradely. However, these matters can be actively withdrawn from the expansion body, for example also with the aid of an aspiration element.

The indwelling venous cannula can have a hollow extension element, a fluid being flowable through the hollow extension element to reduce thrombus formation. If a fluid such as a saline solution flows through the extension element and thus the venous catheter, this prevents the formation of thrombi in the venous catheter that would make it impossible to use the indwelling venous cannula.

However, it is also conceivable that use is made of a mandrin arranged inside the venous catheter, so that thrombus formation is prevented.

It is also conceivable that a mandrin which is arranged inside the venous catheter or can be inserted into said venous catheter is hollow.

The hollow mandrin can exceed a venous catheter in length when it is inserted into said venous catheter. In particular, its near-vein end can project beyond the near-vein end of the venous catheter. This makes it possible to use the hollow mandrin for blood collection and especially for collection of undiluted blood, since the near-vein end of the hollow mandrin comes to lie in a region in which the blood vessel has no contact with the actual venous catheter and/or with possible remnants of infusion solutions, as may be the case inside a venous catheter.

The hollow mandrin can have, at the near-vein end, holes distributed over the circumference for homogeneous supply of a fluid to the living being. Similarly, blood can be collected from the vein via said holes.

The vein-remote end of said hollow mandrin can have a connection element attached thereto. It can have an aspiration element, for example a syringe, connected thereto. Alternatively or additionally, a stop element, for example in the form of a flag, can be attached at this point, so that the hollow mandrin cannot be advanced too far in the longitudinal direction in the direction of the vein or cannot be accidentally lost in the patient.

In principle, the hollow mandrin can also be used as a guide wire. Here, it can have a length of up to 600 cm and can advantageously also be further developed with an internal core. Here, the core, which can be in the form of one part or multiple parts, does not have to be formed over the entire length of the hollow mandrin. The core can completely or partially fill the interior of the hollow mandrin and also consist of multiple parts, sections or components. The core can be removable. The hollow mandrin can thus be used both as a robust guide wire and as a catheter through which fluids can be guided.

The hollow mandrin can also be used as an airway aid, for example as a ventilation tube or as an endotracheal tube. It can also be used as part of tracheotomies or coniotomies. Its near-user end can have a further airway aid, for example a bag valve mask, connected thereto via a connector or without such a connector.

The hollow mandrin can also remain as an inner tube in the venous catheter of the indwelling venous cannula, even over a relatively long period of time. It is then conceivable that it will be replaced regularly in order, for example, to prevent an infection from developing or thrombi from forming.

The hollow mandrin can also be surrounded by a protective cap. The hollow mandrin can be mounted in said protective cap longitudinally slidably or rotatably or completely flexibly.

The protective cap can also be designed in such a way that it has lamellar and/or telescoping properties. In this way, it can be left in place to protect the hollow mandrin during the insertion process and is automatically pushed together when the hollow mandrin passes through the venous catheter, since it cannot pass through it itself.

A protective cap designed in this way can also project beyond the length and circumference of the hollow mandrin in the direction of the vein before the start of the insertion process. This provides an anti-infection guard which protects the patient and user before the start of the intended insertion process. Moreover, the hollow mandrin is thus kept pathogen-free or at least low in pathogens before the start of the intended insertion process.

The protective cap can also be in the form of a flexible protective cover or a bag. The protective cap/protective cover can be tearable or splittable. The protective cap can also encompass only a partial length of the hollow mandrin and be attached to the hollow mandrin slidably in a longitudinal direction. It can then be connected to the hollow mandrin via a loop.

Only when a specific contact pressure is exerted in a specific direction on the near-vein end of the protective cap is it pushed together in the longitudinal direction and, with continued contact pressure, releases an ever greater distance of the hollow mandrin.

Upon removal of the hollow mandrin, the protective cap unfolds again in the longitudinal direction and surrounds all the sections of the hollow mandrin. What therefore arises is a system which is closed outside of the patient/the indwelling venous cannula. This prevents the user from coming into contact with the blood of the patient.

Here, the near-vein end of the protective cap can, for example, be in the form of a container, stamp or bead. A rimlike or ramplike form is also conceivable. A specific form prevents the protective cap from itself not entering the region of the venous catheter during the insertion process.

Here, the near-vein end of the protective cap can be tiltable or designed in such a way that the sides are flexibly movable independently of one another. This makes it possible to introduce the hollow mandrin into the venous catheter from different insertion angles.

The protective cap can also be in the form of a protective cover able to be slid back or other flexible structure. It can also be admixed with or contain a substance which reduces friction, is antimicrobial and/or has a local anesthetic effect. In addition, it can consist of an appropriate material or be provided with an appropriate surface having these properties.

The protective cap can also be in the form of an applicator or sponge or foam ring or gel ring that wets the components of the hollow mandrin to be inserted into the venous catheter, for example with a substance which reduces friction, is antiadhesive, is antithrombogenic, has an antimicrobial effect and/or has a local anesthetic effect. As a result, a venous catheter already in use for a relatively long time, for example, can also advantageously be treated antimicrobially or antithrombogenically from the inside.

The protective cap surrounding the hollow mandrin can also have the properties explained below. It can also surround the puncture needle, the venous catheter and/or all other components of an indwelling venous cannula or else all other catheters and devices.

The indwelling venous cannula can have a safety mechanism which is configured to shield the tip of the puncture needle after the puncture needle has been removed from the venous catheter. Such a shield prevents injury of the user or the living being on the tip of the puncture needle after it is pulled out of the indwelling venous cannula following puncturing.

The puncture needle of the indwelling venous cannula can be surrounded by a protective cap which is removed prior to use of the indwelling venous cannula. In this way, undesired injuries can be avoided when handling the indwelling venous cannula.

It is conceivable that the venous catheter and/or other components of the indwelling venous cannula are also surrounded by a protective cap.

The protective cap can be designed in such a way that it has lamellar or telescopic properties. In this way, the protective cap can be left in place to protect the venous catheter during puncturing and is automatically pushed together during passage of the venous catheter, since it cannot pass through the skin itself during advancement of the venous catheter through the skin.

A protective cap designed in this way can also project beyond the tip of the puncture needle in length and circumference in the direction of the vein before the start of the puncture process. This provides an antipiercing guard which protects the patient and user before the start of the intended puncture process. Moreover, the puncture needle is thus kept pathogen-free or low in pathogens before the start of the intended puncture process.

Only when a specific contact pressure is exerted in a specific direction on the near-vein end of the protective cap is it pushed together in the longitudinal direction and, with continued contact pressure, releases an ever greater distance of the puncture needle or the venous catheter.

Upon removal of the venous catheter, possibly also upon removal thereof with the puncture needle in the event of an unsuccessful puncture, the protective cap unfolds again in the longitudinal direction and surrounds all the sections of the venous catheter and the puncture needle.

Here, the near-vein end of the protective cap can, for example, be in the form of a container, stamp or bead. A rimlike or ramplike form is also conceivable. A specific form prevents the protective cap from getting below the level of the skin during the puncture process.

Here, the near-vein end of the protective cap can be tiltable or designed in such a way that the sides are flexibly movable independently of one another. This makes it possible to introduce the indwelling venous cannula into the patient from different puncture angles.

The protective cap can also be in the form of a protective cover able to be slid back or other flexible structure. It can also be admixed with or contain a substance which reduces friction, is antiadhesive, is antithrombogenic, is antimicrobial and/or has a local anesthetic effect. In addition, it can consist of an appropriate material or be provided with an appropriate surface having these properties.

The protective cap can also be in the form of an applicator or sponge or foam ring or gel ring that wets the components of the indwelling venous cannula to be inserted into the living being, for example with a substance which reduces friction, has an antimicrobial effect and/or has a local anesthetic effect. The use of a puncture-resistant film is possible, too.

The protective cap can also be designed in such a way that it projects beyond only part of the venous catheter and, after puncturing, is pushed in the direction of the vein, for example, in order to accommodate the near-vein end of the venous catheter or the puncture needle in its interior to provide protection. This is especially also relevant in the event of a mispuncture when the indwelling venous cannula containing the venous catheter and puncture needle must be safely disposed of. As already described above, the protective cap can also be combined with a hollow mandrin.

The indwelling venous cannula can have an antipiercing guard for the puncture needle. At least when the indwelling venous cannula is new, the antipiercing guard ensures that self-injury of the user on the tip of the puncture needle cannot occur.

It is conceivable that the antipiercing guard can be designed similarly to the protective cap.

Depth markings can be attached to the venous catheter. The depth markings enable the user to check the insertion depth of the catheter on the patient. In addition, the venous catheter can have a stop ring applied thereto in order to limit the insertion depth of the catheter to a defined distance.

The puncture needle and/or the venous catheter can be rotatably mounted about its own longitudinal axis.

Here, the puncture needle can be freely rotatable about its own longitudinal axis in any position in which it has been slid in the longitudinal direction against the venous catheter. It can be freely rotatable about its own longitudinal axis especially upon complete advancement in the direction of the vein to be punctured.

This makes it possible to spatially align the cut of the near-vein needle tip in relation to the vein to be punctured. However, the puncture needle can also be settable in fixed positions, for example a rotation of 90°, 180°, 270° and/or any rotation from 0° to 360° about its own longitudinal axis, especially upon complete advancement in the direction of the vein to be punctured.

The vein-remote end of the indwelling venous cannula can, for example, contain appropriate devices, for example notches, which interact with a holding element or grip at the vein-remote end of the puncture needle, for example interlock/snap into specific precisely defined positions. It is also possible that a braking effect arises when the puncture needle is in specific positions and/or is rotated about its own longitudinal axis within a specific range of angles. For example, it is possible that greater force from the user is required when rotating the puncture needle within a range from 270° to 360° than when rotating within a range from 0° to 90°. It is also conceivable that the puncture needle automatically comes to a 0° position again and again through a spring mechanism when it is deflected from said position by a user and then released.

The rotatable mounting of the venous catheter can have the advantage that, for example, components of the venous catheter that are obstructed/blocked by anatomical structures, for example entry or exit holes, can be rotated/moved away from the obstructing/blocking structures without having to move or remove the entire indwelling venous cannula.

The puncture needle and venous catheter can also be connected to one another in such a way that they are only rotatably mounted together/in combination.

It is also conceivable that the venous catheter is longitudinally slidably connected to the other components of the indwelling venous cannula or longitudinally slidably mounted in the indwelling venous cannula. Here, the longitudinal slidability can be limited by at least one travel-limiting element. It can have all the properties described in this document with regard to travel-limiting elements. This can have the advantage that the venous catheter can also adapt to further patient movements in a desired end position. Also conceivable is an application such that, before the start of puncturing, the length by which the puncture needle projects beyond the venous catheter near to the vein can be set precisely.

Where angle information is specified in degrees, these are based on a circular measurement of 360 degrees (360°).

The angle information can be marked on a component of the indwelling venous cannula for the user, so that, for example, the current angle of the puncture needle or the venous catheter can be read. Conceivable here are, for example, circular or semicircular markings in different line widths and, for example, also colored markings in the traffic-light colors “green”, “yellow” and “red”.

By way of example, the indwelling venous cannula can be advantageously further developed by the following materials or material classes, substances, elements, etc. (referred to hereinafter as “materials”) individually or in combination. It is possible that all the components of the indwelling venous cannula, especially the venous catheter and/or the puncture needle, are made of or contain the following materials, individually or in combination with one another:
Biocompatible metals and polymers, biopolymers
Biocompatible plastics, explicitly for medical use
Shape memory alloys, for example Nitinol (nickel-titanium alloy)
Copper, zinc, aluminum, iron, tungsten, manganese, silicon, magnesium, cobalt, gold, silver, bronze, platinum, palladium
Brass
Stainless steel, steel alloy, especially also rust-free
Unfired stones
Functional polymers or smart polymers, for example shape memory polymers and thermoresponsive polymers
Ceramic materials, including bioceramics, textile fiber ceramics
Silicone or silicone elastomers
Chromium
Thermoplastics
Thermosets
Elastomers, thermoplastic elastomers (TPE), TPE-A
Polyimides
Polyolefins
Resins and synthetic resins
Rubber, including special-purpose rubber, including EDPM
Vulcanized rubber
Latex
Aramids, aramid fibers, para- and meta-aramid fibers
Cardboard
Natural fibers
Mineral fibers
Fiber composite materials, fiber- and textile-reinforced composites
Carbon fibres which can also reinforce composite materials
Technical textiles
Urethanes, polyurethanes, including thermoplastic polyurethanes (TPU)
Polyester (PES)
Polyvinyl chloride (PVC)- and latex-free materials
Sand, for example quartz sand
Wood
Basalt
Fluoropolymers
Polyurethane elastomers
Thermoplastic high-performance plastics
Polystyrene (PS), including expanded PS
Polyamide (PA)
Polycarbonates (PC)
Perfluoroalkoxy polymers (PFA)
Polysulfone (PSU)
Polyether block amide (PEBA)
Polyetheretherketone (PEEK)
Polyoxymethylene (POM)
Polyphenylene sulfone (PPSU)
Polypropylene (PP)
Polyethylene (PE), including LDPE, HDPE
Polyetherimide (PEI)
Polyethylene terephthalate (PET), Dacron
Polyvinylidene fluoride, polyvinylidene difluo ide (PVDF)
Fluorinated ethylene propylene (FEP)
Polyphenylene sulfide (PPS)
Polyphthalamide (PPA)
Acrylonitrile-butadiene-styrene (ABS)
Methyl methacrylate acrylonitrile butadiene styrene (MABS)
Ethylene vinyl acetate (EVA), EVAC
Elastane
Melamine-formaldehyde resin (MF)
Polyester resin
Phosphorylcholine
Nylon
Teflon
Bentonite
Glass, fiberglass, fiberglass-reinforced plastic
UV glue
PTFE, expanded PTFE, porous PTFE, Polytetraflon® PTFE
ETFE
Parylene
Amino resins
Carbohydrates, sugars
Proteins
Fats
Carbon
Wool
Mylar®, Kapton®, Nomex®, Kevlar®
Web, nonwoven

The use of composite materials and layered composites is possible; hemocompatibility and antiadhesive behavior are advantageous for all materials used. Sterilization resistance and breakage resistance are also advantageous. Advantageously, use can be made of materials having thermoplastic properties and also those that are visible in radiography. UV resistance and high resistance to the effects of chemicals are desirable.

The use of fibers and biodegradable materials is also possible. Films, laminates and/or fabric inserts can used, too.

Resorbable/dissolvable or drug- or matter/substance-delivering materials can also be used. For example, it is possible for materials to release, contain or be coated with substances which are antimicrobial, are anti-inflammatory, have a chemotherapeutic effect and/or have a local anesthetic effect. This also applies, for example, to substances which have an antithrombogenic effect, such as heparin, which can prevent the formation of blood clots on and near the venous catheter.

Surface structure and chemistry of the surface can be optimized. A hydrophilic or hydrophobic coating can be advantageous.

The abovementioned materials can also be used on the venous catheter with local variation. For example, it is conceivable that a different material is used at the end of the venous catheter that is nearest the vein compared to the region that is more remote from the vein. By way of example, the tip of the venous catheter can be made of a softer material having thermoplastic properties. However, the tip of the venous catheter can also be deliberately made of a harder material in order to facilitate advancement into the vein.

Moreover, at one or more points of the indwelling venous cannula, materials can be connected and/or merge into one another by technical processes. For example, it may be possible to connect or fuse a helical spring at the end of the venous catheter that is nearest the vein to a material that surrounds it and lies on the inside or on the outside. Such a connection or fusion can be effected in the region of the entire venous catheter. This can prevent the helical spring from being broken. Advantageously, materials having a suitable toughness can be used here.

Advantageously, materials which expand at body temperature and/or swell, i.e., increase in volume, in the vicinity of a fluid can also be used. For example, bentonite can be used. Such an effect can, for example, be used at the near-vein end of the venous catheter: when the venous catheter is introduced, it is sharp-edged at the near-vein end and has a tight fit with the puncture needle. When the venous catheter is placed in the vein as intended, it now swells at the near-vein end and can thus cause less damage the venous wall. It is also possible that the venous catheter can swell over a further section or over the entire length. It is also possible that the puncture needle can swell as just described and thus changes its properties, for example neutralizes itself and is no longer pointed at the near-vein end. Hydrogels can be used. The attachment of further structures, for example coil-like structures, is also possible.

The use of multiple layers of different materials, which form the wall of the venous catheter for example, is possible, too. Said layers can be fixedly connected to one another or can be slidable against one another. Materials in different states of matter can be present in the wall of the venous catheter.

When using suitable materials, the indwelling venous cannula or components, especially the venous catheter and/or the puncture needle, thereof can be adapted prior to insertion into a patient by the user to the individual course or the individual anatomical characteristics of the vein, for example by bending or deforming.

Refinement of the layers or surfaces mentioned in the last paragraph is possible.

Materials and/or surface coatings can have fluorescent properties.

Materials and/or surface coatings can also change color or change very generally owing to metabolic activities, for example owing to the metabolic activities of pathogens, for example bacteria. However, a change in color or change due to the patient's own metabolic activities is also conceivable. For example, the duration of residence of an indwelling venous cannula or a venous catheter can be determined and monitored.

The material constitution as well can be monitored in this way. It is conceivable that the material properties of the venous catheter change in the event of a relatively long duration of residence or in the event of material defects, for examples breaks or cracks in the venous catheter. For example, the electrical conductivity can change. If a current is now applied across a conductive venous catheter, it can be determined whether the venous catheter is damaged. It is also conceivable that the venous catheter changes properties that make it less visible, more visible or differently visible in an ultrasound image or X-ray image.

Thus, a regular material check can be carried out, especially in the case of venous catheters or indwelling venous cannulas present in the patient for a relatively long time. The duration of residence can thus also be checked. It may also be particularly relevant if the venous catheter has properties that make it less visible, more visible or differently visible in an X-ray check or ultrasound check when pathogens attack. For example, it can be determined whether a venous catheter is the cause of blood poisoning (sepsis). Thus, unnecessary catheter changes can potentially also be avoided.

Advantageously, electrically charged materials can also be used. It is conceivable that the venous catheter is electrically charged, for example negatively charged, owing to specific material properties of its exterior and thus automatically repels itself or moves away from the rather negatively charged inner venous wall (intima). Potential injuries to the venous wall can, then, be avoided through reduced mechanical irritation.

All of the abovementioned materials can also be used on all sections, parts and/or components of the indwelling venous cannula that are configured for dwelling outside of the patient.

The invention will be explained in more detail below on the basis of an exemplary embodiment using a drawing. What is shown is:

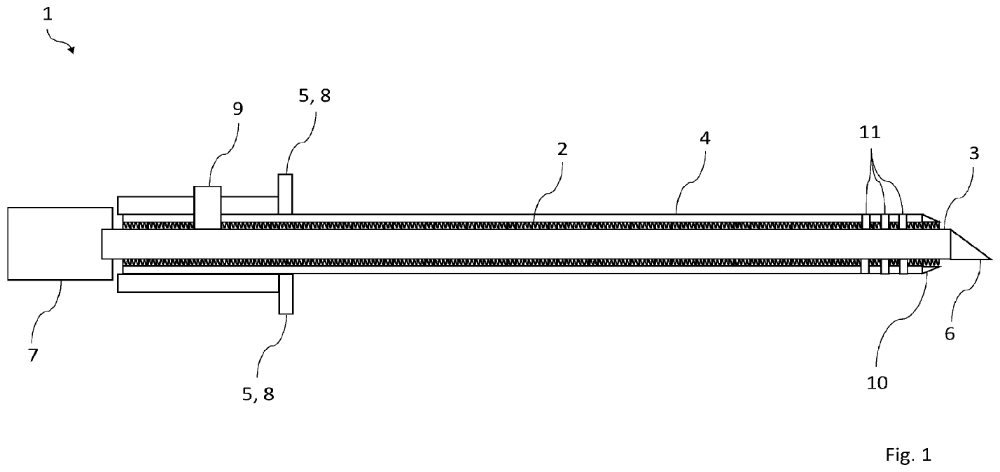

FIG. 1—a schematic depiction of an indwelling venous cannula in a side view.

FIG. 1 shows a schematic depiction of an indwelling venous cannula 1 in a side view. The indwelling venous cannula 1 has a venous catheter 2, and a puncture needle 3 can be longitudinally slidably guided in the venous catheter 2.

The indwelling venous cannula 1 is in the form of a peripheral-vein indwelling cannula 1. It is clear that the venous catheter 2 was formed like a tightly wound helical spring from a puncture-resistant material to give rise to an undulating surface. The venous catheter 2 consists of a puncture-resistant material over its entire length. The helical structure ensures the flexibility of the venous catheter 2. Such a design of the venous catheter 2 provides an antipiercing and anticutting guard which protects the venous catheter 2 from, for example, puncturing by a near-patient tip 6 of the puncture needle 3 during application of the indwelling venous cannula 1. The risk of a damaged venous catheter 2 is thus minimized. It can no longer be sheared off by the near-patient tip 6 of the puncture needle 3, even with repeated sliding of the puncture needle 3 relative to the venous catheter 2. Scrap due to damaged venous catheters can thus be distinctly reduced. Moreover, repeated use of the indwelling venous cannula 1 as part of a puncture process under constant sterile conditions on a living being is possible. This is especially important if mispuncture initially occurs, i.e., the blood vessel was accidentally missed during the first puncture or the venous catheter 2 could not initially be advanced far enough into the blood vessel.

For improved aspiration, the venous catheter 2 is provided with a sealing coating 4. The undulating surface can give rise to minimal openings which make aspiration difficult, since it is through said openings that, for example, air can be undesirably drawn. A sealing coating 4 can minimize or prevent the undesired aspiration of air. Advantageously, the sealing coating 4 is a PTFE coating which, at the same time, facilitates the insertion of the venous catheter 2 into the punctured body part. An additional dilation element 10 at the near-patient end of the venous catheter 2 achieves uniform expansion when the venous catheter 2 is advanced in the punctured body part.

The application of the indwelling venous cannula 1 to a living being can, for example, proceed in the following steps:
1. Puncturing a vein using the puncture needle 3
2. Advancing the helical venous catheter 2 having the sealing coating 4 over the near-patient tip 6 of the puncture needle 3 into the vein to the desired position
3. Removing/sliding back the puncture needle 3
4. Closing the helical venous catheter 2 at a patient-remote end.

The indwelling venous cannula 1 has two holding elements 5. Said holding elements 5 enable the user to operate the indwelling venous cannula 1 with one hand, the second hand being able to be used, for example, to stabilize the body part to be punctured. The puncture needle 3 is in the form of a hollow needle. After puncturing by the near-patient tip 6 of the puncture needle 3, the user can directly identify whether the vein has been punctured correctly, in that the hollow puncture needle 3 fills with venous blood and enters the chamber 7, whereby the blood can be directly perceived by the user.

After puncturing, the venous catheter 2 can be slid into the punctured body part and the puncture needle 3 together with the chamber 7 can immediately be pulled out of the components of the indwelling venous cannula 1 that remain in the body part. A safety mechanism can be designed so that the near-patient tip 6 of the puncture needle 3 is shielded after it has been pulled out of the indwelling venous cannula 1 and thus protects the user and the living being from possible stab injuries.

In its end position in the punctured body part, the venous catheter 2 can be held fixed to the living being via fastening elements 8. The fixation can be done by means of a self-adhesive wound dressing, which fixes the indwelling venous cannula 1 to the living being via the fastening elements 8. The fastening elements 8 described, which can be in the form of wings for example, are optional elements of the indwelling venous cannula 1.

Here, the puncture needle can run essentially in the middle between the holding elements 5 and/or the fastening elements 8.

It is clear that the function of the holding element 5 and the fastening element 8 can be combined in one element. This allows simple production of the indwelling venous cannula 1, while at the same time the structure of the indwelling venous cannula 1 is kept simple for the user.

An aspiration element such as a syringe can be connected via a connection element 9. The connection element 9 can be in the form of a valve which allows simple drug administration or aspiration of blood. Here, when the valve is untouched, it prevents liquids, such as blood, from running retrogradely out of the connection element 9. Moreover, when the valve is untouched, it prevents air from penetrating into the connection element 9 from the outside. The connection element 9 can moreover contain a filter which prevents coarse particles, bacteria and air from penetrating into the interior of the connection element 9 and thus into the interior of the indwelling venous cannula.

An aspiration element such as a syringe can be connected to the chamber 7. As a result, the indwelling venous cannula can be inserted into a vein under continuous aspiration using the syringe. The success of puncture can therefore be established directly and very precisely. Here, chamber 7 can also be in the form of a further valve which allows the flow of fluids in only one defined direction. Moreover, chamber 7 can alternatively or additionally also be designed in such a way that it prevents air from penetrating or only allows passage of air and other gases and vapors in one defined direction. The chamber 7 can, for example, be designed in the same way as the connection element 9.

The chamber 7 and the connection element 9 can be covered by a protective cap, so that undesirable contamination does not occur when the chamber 7 and the connection element 9 are not in use. The protective cap can be connected to the chamber 7 via a tab and/or to the connection element 9 via a tab.

It is also conceivable that when a protective cap, which can be connected to the connection element 9 or to the chamber 7 via a tab, is pressed down, the fluid flow in the indwelling venous cannula is interrupted or slowed down.

At the near-patient end, the venous catheter 2 has holes 11 distributed over the circumference. It is through the holes 11 that a homogeneous delivery of, for example, a drug into the living being can be achieved. An undesired, locally highly concentrated delivery of the drug into the living being is thus avoided. By arrangement of a plurality of holes 11, it is moreover possible to increase the flow rates of the administered infusion solutions and drugs. Moreover, this can facilitate aspiration of liquids, such as blood collection, from the living being via the situated venous catheter or via the situated indwelling venous cannula. With appropriate construction of other components of the indwelling venous cannula, what may also be possible as a result is fluids, vapors and/or gases spontaneously escaping from the indwelling venous cannula in a desired and retrograde manner, if it is to be used, for example, for drainage of fluids, vapors and/or gases, for example as part of a puncture of the pleural space, other cavities or the above-described body spaces.

What is presently depicted is merely a schematic depiction giving a good overview of the components of the indwelling venous cannula according to the invention. However, the lengths and proportions can differ in reality.

FIG. 1 is one possible exemplary embodiment. Other forms of the teaching according to the invention are also conceivable. Furthermore, the embodiments of the exemplary embodiment are not inseparably linked to one another, and so, for example, the implementation of the invention is not dependent on the embodiments of the exemplary embodiment that have been specifically described. Thus, variability of, for example, the number, length or size of the individual elements is always conceivable.

### LIST OF REFERENCE SIGNS

1—Indwelling venous cannula
2—Venous catheter
3—Puncture needle
4—Sealing coating
5—Holding element
6—Tip
7—Chamber
8—Fastening element
9—Connection element
10—Dilation body
11—Holes

1. An indwelling venous cannula for application to a living being, comprising:
a venous catheter; and
a puncture needle which is longitudinally slidably guidable in the venous catheter, wherein the puncture needle is in a form of a hollow needle over part of its length, wherein another part of the length of the puncture needle is at least partly solid.

2. The indwelling venous cannula as claimed in claim 1, wherein the venous catheter is made of a puncture-resistant material or is coated with the puncture-resistant material.

3. The indwelling venous cannula as claimed in claim 1, wherein the venous catheter is made of a self-closing material or material layers.

4. The indwelling venous cannula as claimed in claim 1, further comprising at least one holding element for facilitated application to the living being, wherein the holding element has a complementary shape or a negative contour of a human thumb or a human fingertip.

5. The indwelling venous cannula as claimed in claim 1, further comprising a fastening element, wherein the fastening element is configured to fix the indwelling venous cannula to the living being, wherein the fastening element has an adherent or adhesive surface or an adherent or adhesive coating for fixation of the indwelling venous cannula to the living being.

6. The indwelling venous cannula as claimed claim 1 wherein the venous catheter and/or the puncture needle has an undulating surface.

7. The indwelling venous cannula as claimed in claim 6, wherein the venous catheter and/or the puncture needle has a helical structure and/or is provided with a helical structure, wherein the helical structure has windings, wherein the density of the windings varies over the length of the venous catheter and/or the puncture needle.

8. The indwelling venous cannula as claimed in claim 1 wherein the venous catheter has, at a near-patient end, a dilation body for uniform expansion of a punctured body part.

9. The indwelling venous cannula as claimed in claim 8, wherein a diameter of the dilation body increases from the near-patient end along the dilation body.

10. The indwelling venous cannula as claimed in claim 9, wherein the diameter of the dilation body increases stepwise.

11. The indwelling venous cannula as claimed in claim 1 wherein the venous catheter has a connection element for connection of an aspiration element, wherein the venous catheter has a structure configured for locking with the aspiration element.

12. The indwelling venous cannula as claimed in claim 1 wherein a near-vein end of the puncture needle or a vein-remote end of the puncture needle is hollow.

13. The indwelling venous cannula as claimed in claim 12, wherein the vein-remote end of the puncture needle is hollow and is connected to at least one hole, wherein the at least one hole is arranged laterally on the puncture needle.

14. The indwelling venous cannula as claimed in claim 1 wherein the puncture needle and the venous catheter are curved and/or angled and/or helical.

15. The indwelling venous cannula as claimed in claim 1 wherein in the venous catheter is layered, wherein individual layers are detachably arranged in a lumen of the venous catheter.

16. The indwelling venous cannula as claimed in claim 1 wherein at least part of the venous catheter comprises an aramid and/or aramid fibers.

17. The indwelling venous cannula as claimed in claim 1 wherein at least part of the venous catheter and/or the puncture needle comprises a resorbable material or at least part thereof is coated with the resorbable material.

18. The indwelling venous cannula as claimed in claim 17, wherein at least part of a tip of the puncture needle consists of a resorbable material and/or is coated with a resorbable material.

19. The indwelling venous cannula as claimed in claim 1 wherein the venous catheter is stretchable in relation to its diameter.

20. The indwelling venous cannula as claimed in claim 1 further comprising a clamping element arranged on the venous catheter, wherein the clamping element is configured to interrupt a fluid flow through the venous catheter.

21. The indwelling venous cannula as claimed in claim 1 further comprising a sensor at a near-vein end.

22. The indwelling venous cannula as claimed in claim 1 wherein the venous catheter and/or the puncture needle has a helical structure and, wherein the puncture needle is detachably connected to the venous catheter.

23. The indwelling venous cannula as claimed in claim 1 wherein the venous catheter is multiluminal.

24. The indwelling venous cannula as claimed in claim 23, wherein at least one lumen is configured for rinsing of the venous catheter of the living being.

25. The indwelling venous cannula as claimed in claim 1 wherein the puncture needle and/or the venous catheter is rotatably mounted about its own longitudinal axis.
